(19) United States
(12) Patent Application Publication
(10) Pub. No.: US 2024/0188976 A1
(43) Pub. Date: Jun. 13, 2024

(54) DEVICES AND METHODS FOR RESTRICTING FLOW IN VESSELS AND REMOVING MATERIAL FROM VESSELS

(71) Applicant: Penumbra, Inc., Alameda, CA (US)

(72) Inventors: Jorge Gamba, Jacksonville, FL (US); Eric Sauvageau, Jacksonville, FL (US); Michael Schaller, Louisville, CO (US)

(73) Assignee: Penumbra, Inc., Alameda, CA (US)

(21) Appl. No.: 18/533,678

(22) Filed: Dec. 8, 2023

Related U.S. Application Data
(60) Provisional application No. 63/431,127, filed on Dec. 8, 2022.

(51) Int. Cl. A61B 17/221; A61M 25/00

(52) U.S. Cl. CPC A61B 17/221; A61M 25/0074; A61M 25/0082; A61B 2017/22079

(57) ABSTRACT

A device for removing material from a patient includes a catheter having a distal catheter end and defining a catheter lumen and a containing element coupled to the catheter. The containing element is deployable from the distal catheter end of the catheter and includes an outer wall portion. The outer wall portion includes an inner layer defining an inner chamber and an outer layer coupled to the inner layer. The inner layer and the outer layer form a distal folded edge defining a distal opening of the containing element in communication with the inner chamber. Implementations of the device may further include a control element extending through the catheter lumen and coupled to the containing element for selectively collapsing the inner chamber, a membrane for restricting flow through the outer wall portion, and/or a flow restriction formed by the containing element.

### CROSS-REFERENCE TO RELATED APPLICATIONS

This application claims the priority benefit of U.S. Provisional Patent Application No. 63/431,127 filed Dec. 8, 2022, titled “Devices and Methods for Restricting Flow in Vessels and Removing Material from Vessels”, the entirety of which is incorporated herein by reference.

### TECHNICAL FIELD

This disclosure relates generally to the field of surgery, and more specifically to the field of interventional radiology. Described herein are devices and methods for removing material from a patient.

### BACKGROUND

Minimally invasive endovascular techniques have come to the forefront in the safe and expeditious use of embolectomy devices for thromboembolic clot extraction. This includes, without limitation, clot extraction to treat neurovascular ischemic stroke, pulmonary embolism, deep vein thrombosis, arterial thrombosis, stone removal, and others. Currently employed devices generally extract the clot using a combination of balloons, graspers, aspiration, and wire retrievers. Many of these devices attempt to remove the clot in vivo by attaching to it and then pulling it through the vascular lumen and out of the body. With these devices the thrombus is typically not fully contained and if fragments of the clot break away, they may become new emboli in the blood stream. That is to say that existing devices typically maintain partial or full exposure of the thrombus within the vascular lumen and when clot extraction is attempted, the “bare thrombus” can pose a threat of fragmentation or partial clot dislodgement which can predispose a patient to inadvertent distal embolization, non-target territory embolization or incomplete thrombus extraction.

Additionally, in order to limit the blood flow in the clotted vessel during clot removal, many procedures utilize a variety of flow arrest techniques such as balloon-assisted proximal vessel occlusion to minimize antegrade flow in an effort to exclude distal clot fragmentation during clot extraction. Mechanical or assisted suction techniques are oftentimes utilized simultaneously via the balloon flow arrest catheter to capture any potential embolic debris during clot extraction. However, some existing flow reducing devices such as balloon guide catheters are inherently stiff and difficult to deliver to their target location and are often larger than desired, requiring large entry wounds to access the vasculature. Additionally, in some applications, complete flow arrest is often difficult due to extensive collaterals, such as with neuro thrombectomy and the collateral intracranial vessels (e.g., Circle of Willis), limiting the efficacy and utility of proximal flow arrest and suction in the carotid circulation. Even limited blood flow can create a significant risk of clot fragmentation and distal migration of clot during extraction.

Encasing the occlusive material during removal from the patient's vasculature and providing flow arrest in the vessel during material removal would potentially improve patient outcomes.

### SUMMARY

In one aspect of this disclosure, a device is provided. The device includes a catheter having a distal catheter end and defining a catheter lumen and a containing element coupled to the catheter. The containing element is configured to be deployed from the distal catheter end of the catheter and includes an outer wall portion. The outer wall portion includes an inner layer defining an inner chamber in communication with the catheter lumen and an outer layer coupled to the inner layer. When the containing element is deployed from the catheter, the inner layer and the outer layer form a distal folded edge defining a distal opening of the containing element in communication with the inner chamber. The device further includes a control element extending through the catheter lumen and coupled to the containing element. The control element is longitudinally movable relative to the catheter to selectively collapse the inner chamber defined by the inner layer when the containing element is deployed.

In another aspect of this disclosure, an alternative device is provided. The device includes a catheter having a distal catheter end and defining a catheter lumen and a containing element coupled to the catheter. The containing element is configured to be deployed from the distal catheter end of the catheter and includes an outer wall portion. The outer wall portion includes an inner layer defining an inner chamber in communication with the catheter lumen and an outer layer coupled to the inner layer. When the containing element is deployed from the catheter, the inner layer and the outer layer form a distal folded edge defining a distal opening of the containing element in communication with the inner chamber. The outer layer is further configured to form a proximally concave surface when the containing element is deployed from the distal catheter end.

In yet another aspect of this disclosure, a system is provided. The system includes an aspiration source, a delivery catheter, and a catheter disposed within the delivery catheter. The catheter includes a distal catheter end and defines a catheter lumen in fluid communication with the aspiration source. The system further includes a containing element coupled to the catheter. The containing element is configured to be deployed from the distal catheter end of the catheter and includes an outer wall portion. The outer wall portion includes an inner layer defining an inner chamber of the containing element in communication with the catheter lumen and an outer layer coupled to the inner layer. When the containing element is deployed from the catheter, the inner layer and the outer layer form a distal folded edge defining a distal opening of the containing element in communication with the inner chamber. The outer wall portion further includes a membrane configured to restrict flow through the outer wall portion. The system also includes a control element extending through the catheter lumen and coupled to the containing element. The control element is longitudinally movable relative to the catheter to selectively collapse the inner chamber defined by the inner layer when the containing element is deployed.

The foregoing is a summary and may be limited in detail. The above-mentioned aspects, as well as other aspects, features, and advantages of the present technology are described below in connection with various implementations, with reference made to the description, claims and accompanying drawings.

### BRIEF DESCRIPTION OF THE DRAWINGS

FIG. 1A is an oblique view of a first embodiment of a device according to the present disclosure.

FIG. 1B is a detailed view of a containing element distal opening of the device of FIG. 1A.

FIG. 2 is a side view of the device of FIG. 1A with the containing element coupled to a catheter.

FIGS. 3A-3D illustrate example operation of the device of FIG. 1A and, more specifically, controlled collapsing of a volume of the containing element of the device.

FIG. 4A illustrates the device of FIG. 1A with the outer layer and inner layer unfolded with a membrane applied to the inner layer, such as may occur during assembly of the device.

FIG. 4B illustrates the device of FIG. 1A with the outer layer and folded over the inner layer.

FIG. 5 illustrates an alternate implementation of a device according to the present disclosure with a passive interior chamber distal restriction.

FIG. 6 illustrates an alternate implementation of a device according to the present disclosure with a passive interior chamber distal restriction and a shortened inner layer.

FIG. 7 illustrates an alternate implementation of a device according to the present disclosure with a filament.

FIG. 8 illustrates an alternate implementation of a device according to the present disclosure with an angled distal opening.

FIG. 9A illustrates an alternate implementation of a device according to the present disclosure with a vessel sealing surface.

FIG. 9B illustrates an alternate implementation of a device with a vessel sealing surface.

FIG. 9C illustrates an implementation of the device with a vessel sealing surface in a vessel.

FIG. 10A illustrates an alternate implementation of a device according to the present disclosure deployed in a vessel.

FIG. 10B illustrates the device of FIG. 10A with a vessel sealing surface.

FIG. 10C illustrates the device of FIG. 10A with the vessel sealing surface further formed.

FIG. 11A illustrates an alternate implementation of a device according to the present disclosure with a vessel sealing surface and a container.

FIG. 11B illustrates an alternate implementation of a device according to the present disclosure with a vessel sealing surface and a container.

FIG. 12A illustrates an alternate implementation of a device according to the present disclosure with a vessel sealing surface.

FIG. 12B illustrates the device of FIG. 12A in a vessel.

FIG. 13A illustrates an alternate implementation of a device according to the present disclosure with a vessel sealing surface.

FIG. 13B illustrates the device of FIG. 13A in an unfolded state.

FIG. 14 illustrates an alternate implementation of a device according to the present disclosure with a vessel sealing surface.

### DETAILED DESCRIPTION

The present disclosure is directed to devices and methods for removing material from a blood vessel and restricting blood flow that address various shortcomings of existing and conventional devices. In one example application, the devices and methods are used to capture and remove an occlusive clot from a patient's vasculature. The device includes a containing element which is constricted and which is advanced to a vascular location. The containing element is then deployed in a position to receive and contain material for removal. A clot retrieving element such as a stent retriever or aspiration may be used to engage the material to be removed and assist in moving the material into the containing element.

The containing element has a distal opening at a distal end and an outer wall portion extending proximally from the distal opening. The distal opening may be moved to an open position to receive the material in an interior chamber formed by the outer wall. The device may include a suction source connected to an aspiration path that runs through a delivery catheter, through the containing element, and through the distal opening of the containing element into the vessel.

The outer wall portion of the containing element may be formed of a braided material with an inner layer and an outer layer where the inner layer and outer layer are connected at a folded edge at the distal opening. The outer wall portion further may further include a membrane that restricts blood flow in a vessel.

Once the material is contained within the containing element, the containing element can be moved to a closed configuration in which a distal restriction reduces the internal diameter of the interior chamber at a location to prevent material from escaping through the distal opening as the containing element is removed and/or moved into another catheter or sheath for removal from the patient.

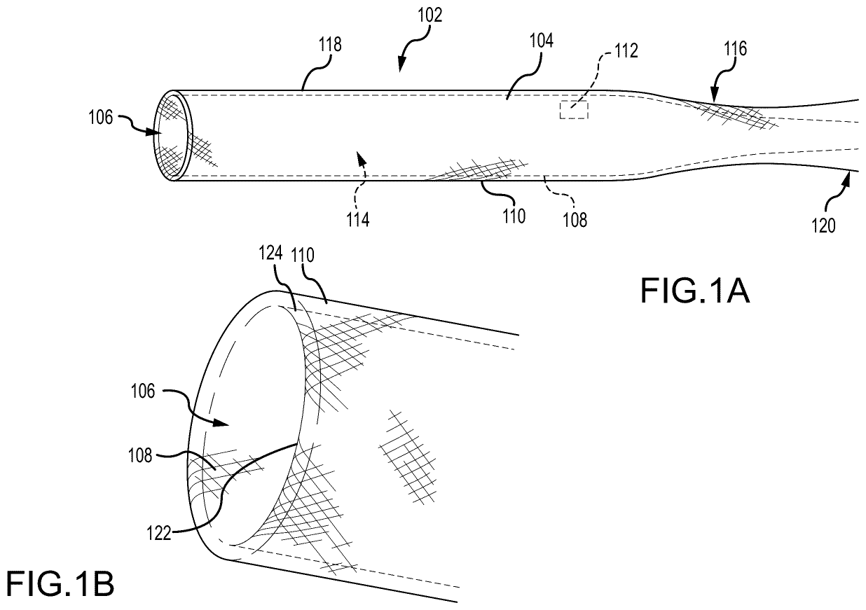

In FIG. 1A, a device 102 to remove material from a blood vessel and/or restrict blood flow in the blood vessel is shown. The device 102 includes a containing element 104 with an outer wall portion 118 which forms an interior chamber 114 and defines a distal opening 106. The outer wall portion 118 may have a multi-layer construction including an inner layer 108 and an outer layer 110. The outer wall portion 118 may also optionally include a membrane 112 for restricting or fully obstructing flow through the outer wall portion 118. The containing element 104 further has a tapered region 116 and a proximal end 120. The device 102 is shown in a generally expanded configuration with the containing element 104 unrestricted.

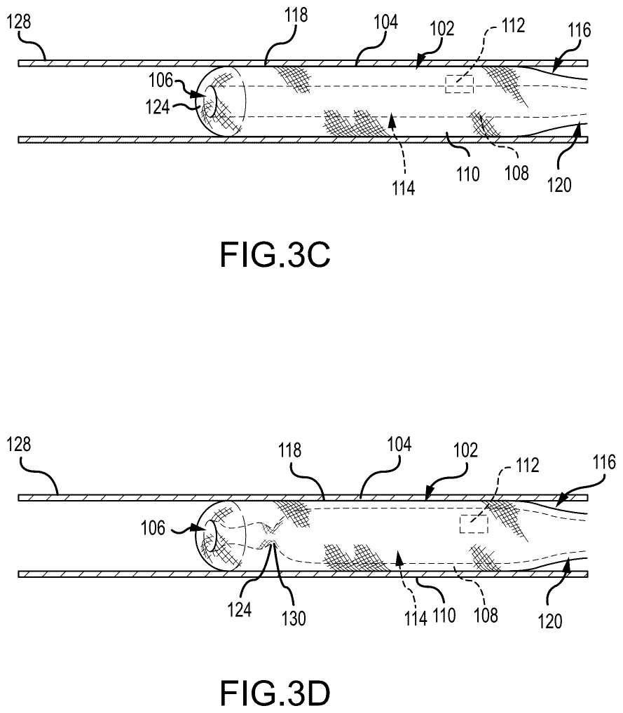

For clarity, FIG. 1A illustrates only a portion of the membrane 112. As discussed later in this disclosure, in certain implementations the membrane 112 may be a semi-permeable or impermeable layer of material configured to prevent or restrict fluid flow through the outer wall portion 118 of the containing element 104. The membrane 112 may be disposed in various locations of the outer wall portion 118. For example, the membrane 112 may be coupled to an interior surface or exterior surface of either of the inner layer 108 or the outer layer 110 or may be suspended between the inner layer 108 and the outer layer 110. In general, devices according to this disclosure may include any suitable membrane or multiple membranes with different properties. For clarity and simplicity, membranes of this disclosure are only shown in part, such as the membrane 112 in FIG. 1A. An example of a full membrane is shown in FIG. 4A.

In FIG. 1B, a detailed view of a distal end of device 102 is shown. The inner layer 108 connects to the outer layer 110 at a folded edge 124 which defines at least a portion of the perimeter of the distal opening 106. As shown, the folded edge 124 may form a series of loops 122 at the distal opening 106 which are atraumatic and which can be connected to various other devices.

The various construction and elements of an implementation of the device 102 will be described in greater detail below. Aspects of the present invention are described with reference to a single or limited number of implementations; however, it is understood that all features, aspects, and methods are incorporated into all applicable implementations described herein even though not expressly mentioned or set forth. For example, the detailed description below is primarily directed to neurothrombectomy applications, with the example implementation of the device 102 including dimensions such as diameters, lengths, thicknesses etc., suitable for such applications. In other applications these dimensions may be different depending on factors, such as but not limited to, the target vessel size and clot size. Any ranges provided in the application are exemplary only and should not limit the scope or application of the device or methods described in this disclosure.

In FIG. 1A, a part of device 102 is shown with the distal opening 106 expanded. The containing element 104 is an expandable tube that can be made partly of a metallic braid, such as nitinol, stainless steel, cobalt-chromium, or any other suitable biocompatible material. Alternatively, the tube can be made from a polymeric braid, such as polyester, polypropylene, or any other suitable material. Although the term braid is used herein, other terms such as weave, scaffold, structure, fabric can be used and should not limit the scope of the disclosed device. In some implementations, the expandable tube can be comprised of laser cut material similar to that used in stent manufacturing. Additionally, the tube does not necessarily need to be a contiguous cylinder and can in fact have other structures such as, but not limited to, a rolled-up tube made from similar components described herein.

Various characteristics and dimensions of the device 102 may vary depending on the specific application of the device 102. For example, in certain implementation, the braid of the inner layer 108 and/or the outer layer 110 can be formed from woven wires. The specific number of wires may vary, however, in certain implementations, the number of wires may be from and including about 8 wires to and including about 288 wires or from and including about 72 to and including about 144 wires or about 96 wires. Wire diameter may similarly vary based on application. Nevertheless, in certain non-limiting implementations, the wire diameter may be from and including about 0.0001″ to and including about 0.008″ or from and including about 0.0005″ to and including about 0.0020″ or about 0.0010″. As another example, the weave pattern as defined by a braid angle may also vary. However, in certain non-limiting examples, the braid angle may be from and including about 80 degrees to and including about 160 degrees, from and including about 100 degrees to and including about 140 degrees, or about 120 degrees. Notably, the braid may vary in any of the foregoing aspects or other aspects along its length. For example, the braid angle can be about 100 degrees in some areas and can be about 120 degrees in other areas. The braid may even include wires of various wire diameters. For example, a first set of wires forming the braid may have a wire diameter of about 0.0010″ while a second set of wires forming the braid may have a wire diameter of about 0.0015″. In still other implementations, the inner layer 108 may have different characteristics than the outer layer 110. For example, the outer layer 110 may have higher density of braid wires with smaller wire diameters whose primary purpose is to cover a membrane 112 between the two layers, while the inner layer 108 may have fewer wires but with larger wire diameters to increase opening radial force and maintain a patent interior chamber under aspirational vacuum forces. This disclosure contemplates that the device 102 may also include any other variations in overall dimensions, weave parameters, wire characteristics, and the like while being within the scope of this disclosure.

Referring to FIG. 1A and FIG. 1B, the outer layer 110 of the braided material forming the containing element 104 extends along the length of the containing element 104 toward the distal opening 106 where it is everted or inverted such that an inner layer 108 is formed that then runs proximally toward the proximal end 120 of the containing element 104, thereby forming the interior chamber 114. Accordingly, the interior chamber 114 is formed by the outer wall portion 118 which in at least one implementation has at least two generally concentric layers. The folded edge 124 (shown in FIG. 1B) is formed at the juncture between the inner layer 108 and the outer layer 110 with a series of loops 122 formed about the perimeter of the folded edge 124. In certain implementations, the inner layer 108 may have a profile that generally matches the outer layer 110 as shown in FIG. 1A, which illustrates both the inner layer 108 and the outer layer 110 having a tapered region 116. In other implementations, the inner layer 108 and the outer layer 110 may have different profiles. For example, the outer layer 110 may generally form a cylinder that opposes the vessel wall while the inner layer 108 may have features that restrict the distal end of the interior chamber and therefore are not matched to the outer layer 110. Examples of such implementations are be described in more detail, below. In still other implementations, the inner layer 108 may have a tapered region 116 which does not match the tapered region of the outer layer 110 or the outer layer 110 may not have a tapered region 116 at all.

In certain implementations, the containing element 104 can be made by first making a single tube of braided wire such as a Nitinol wire. The diameter of the tube may be sized for its application and can therefore vary. By way of non-limiting example, in the case of neurothrombectomy, the tube used to form the containing element 104 can have an outer diameter from and including about 2 mm to and including about 7 mm or about 5 mm. The tube can then be inverted by taking the distal end of the tube and either rolling the distal end outwardly along the tube or by folding the distal end into the tube body. Either way, such rolling results in the formation of the outer layer 110 and the inner layer 108, which are generally concentric with one another.

Following initial formation of the inner layer 108 and the outer layer 110, the inner layer 108 and the outer layer 110 may be opposed to one another using a mandrel on the inside diameter of the inner layer 108 and a sleeve or mold on the outside diameter of the outer layer 110. In this step, the folded edge 124 can also be formed where the inner layer 108 and the outer layer 110 meet. Additional profiles to the inner layer 108 or the outer layer 110 can be applied at this stage as well, e.g., using the shape and profile of the mandrel or the sleeve. For example, either the mandrel or the sleeve or both can include a profile for creating the tapered region 116 of the containing element 104. Any number of other profiles are contemplated herein.

In the implementation where the inner layer 108 or the outer layer 110 are a braid constructed of nitinol wires or a similar shape settable material (e.g., a heat-settable material), the containing element 104 can be placed in a kiln or otherwise heat-treated to shape set the braid in the predetermined shape defined by either the mandrel or sleeve or both. In general, setting (e.g., by heating or another similar setting process) may be used to cause the containing element 104 to maintain a predefined shape when deployed. Setting may be applied generally to the containing element 104 or may be applied selective to specific areas, such as to the distal folded edge 124. Once the containing element 104 has its predetermined shape, the mandrel and sleeve can be removed. Alternatively, the braid material may be stainless steel or other a similar material that can be plastically deformed by either the mandrel or sleeve or both, obviating the need for a shape setting step.

The membrane 112 can be a part of at least a section of the outer wall portion 118. In some implementations, the membrane 112 is sandwiched between the inner layer 108 and outer layer 110. The membrane 112 can, for example, be attached onto the outer circumference of the inner layer 108 which is then covered by the outer layer 110 when the outer layer 110 is made to extend back over the inner layer 108 (e.g., by rolling the inner layer 108 into the braided tube or rolling the outer layer 110 around an outside of the braided tube). Covering the membrane 112 with the outer layer 110 may be advantageous because the membrane 112 may have higher friction against the lumen of a delivery catheter which can result in difficulty delivering the device 102 to a clot through a tortuous vessel. By adhering the membrane 112 to the inner layer 108, the membrane 112 may be prevented from significantly rubbing against the lumen of the delivery catheter because it is covered by the outer layer 110.

In other implementations, the membrane 112 can be attached to the inner lumen of the outer layer 110 and, therefore, still sandwiched between the two layers. In still other implementations, the membrane 112 can be attached to the inner lumen of the inner layer 108. In still other implementations, the membrane 112 can be attached to the outer circumference of the outer layer 110. The membrane 112 can also be attached to multiple locations. In some implementations, the membrane 112 is sandwiched between the inner layer 108 and outer layer 110, while being laminated to both layers.

This disclosure also contemplates that the device 102 may include multiple membranes disposed, with each membrane disposed to any of the arrangements noted above.

Lubricious or low friction coatings may additionally be employed in any component within device 102. For example, the membrane 112, the inner layer 108, the outer layer 110, or the catheter 126 may include hydrophilic coatings or other lubricious elements to facilitate delivery and device 102 use.

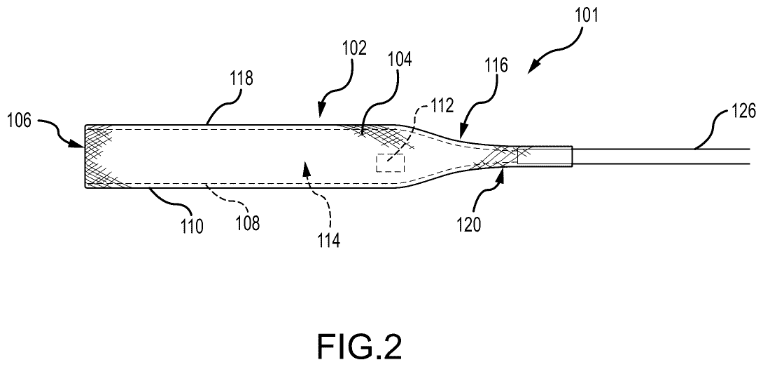

FIG. 2 illustrates a catheter-based device 101 including the device 102. As shown in FIG. 2, the catheter-based device 101 is assembled by coupling the device 102 to a catheter 126 such that the containing element 104 of device 102 is attached at its proximal end 120 to the catheter 126. The catheter 126 can be manufacturing via any suitable catheter manufacturing techniques and may vary in construction, dimensions, and materials. In the specific example shown in FIG. 2, the catheter 126 is formed such that it includes an internal lumen with a sidewall. By way of non-limiting example, the sidewall may be formed of liner, internal braid or coil, and jacket. For example, the catheter 126 may include a PTFE or similar material liner with a stainless-steel braid or coil and an external Pebax jacket. Any number of other catheter manufacturing techniques and materials may be employed.

As with device 102, the catheter 126 may be sized according to its clinical application. For example, the catheter 126 may be sized such that it can be delivered through a delivery catheter, which, in turn may be suitably sized for the relevant vasculature. In the example of neurovascular thrombectomy for ischemic stroke, for example, the delivery catheter can have an inner diameter from and including about 0.044″ to and including about 0.100″, from including about 0.056″ to and including about 0.088″, or about 0.071″. Consequently, in such implementations, the catheter 126 can have an inner diameter from and including about 0.014″ to and including about 0.071″, from and including about 0.035″ to and including about 0.056″, or about 0.044″, respectively, but not larger than the inner diameter of the delivery catheter when combined with the wall thickness of the catheter 126. The outer diameter of the catheter 126 can be sized generally to be less than the inner diameter of the delivery catheter. The catheter 126 can be long enough to extend through the entire lumen of the delivery catheter and therefore, in the case of neurovascular thrombectomy. For example, the length of catheter 126 may be from and including about 80 cm to and including about 200 cm, from and including about 100 cm to 160 cm, or about 140 cm with the length of the delivery catheter being shorter than the length of the catheter 126. In other implementations, the catheter 126 can be shorter such that it does not stick out the end of the delivery catheter and instead transitions to an element such as a wire that can control the translation of the containing element through the delivery catheter and in the vessel.

In implementations in which the membrane 112 is at least partially attached to either the inner layer 108 or outer layer 110, the membrane 112 can be further attached to the catheter 126. For example, the membrane 112 may be partially coupled to the inner layer 108 or the outer layer 110 and may extend proximally to the catheter 126. The membrane 112 may then be coupled to either the interior or exterior surface of the catheter 126. Among other uses, doing so can fluidly connect the lumen of the catheter 126 to the interior chamber 114 of the containing element 104 such that any aspiration applied to the catheter 126 goes through the containing element 104 as well and does not leak. When expanded within a vessel as discussed below, this can allow aspiration applied to the catheter 126 to be applied to the vessel directly.

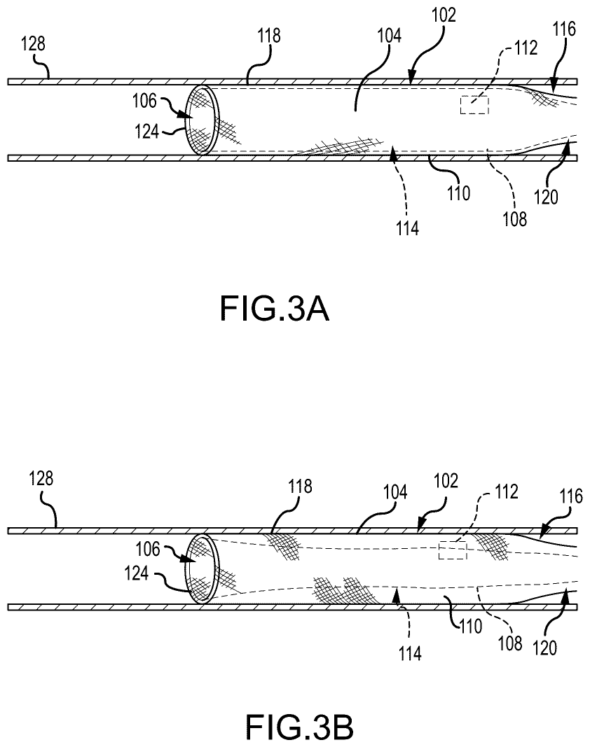

FIGS. 3A-3D illustrate operation of the device 102 and, in particular, controlled collapsing and constriction of the inner layer 108, as would be performed when retaining embolic or other material. In FIG. 3A, the device 102 is shown in an expanded configuration within a vessel 128, e.g., following deployment into the vessel 128 through a delivery catheter. In the illustrated example, the outer layer 110 is rigidly connected to the catheter 126 (not shown but positioned proximal the device 102). For example, the outer layer 110 may be bonded to the tip or outer diameter of the catheter 126. The inner layer 108 is not rigidly connected to the catheter 126 and instead is connected to a control body which can manipulate the location of the inner layer 108. In some implementations, the inner layer 108 enters into the lumen of catheter 126 and then is attached to the control body. The control body may be a wire or a tube that is attached to the proximal end 120 of the inner layer 108. With the device 102 in an expanded configuration, the membrane 112 of the containing element 104 can reduce or entirely stop blood flow through the vessel 128. In the expanded configuration, the distal opening 106 is generally open and a clot can be urged into the interior chamber 114 using either aspiration or a device such as a stent retriever, or any other suitable method. For example, using an aspiration source attached to the catheter 126 the user can suck the clot into the interior chamber 114 since blood flow is arrested in the vessel. With the clot inside the interior chamber 114, the distal end of the containing element 104 can now be closed to prevent the unwanted removal of the clot from the interior chamber 114.

In FIG. 3B, the proximal end 120 of the inner layer 108 has been moved proximally by a proximally directed force from a control body such that tension is applied to the inner layer 108. As can be shown, the inner layer 108 stretches and thus reduces in diameter. This separates the outer layer 110 and inner layer 108 such that they are not directly in contact. With a clot inside the interior chamber 114, the inner layer 108 can reduce around the clot and contain it.

In FIG. 3C, the proximal end 120 of the inner layer 108 has been moved further proximally and the interior chamber 114 defined by the inner layer 108 and membrane 112 has been reduced further. As can also be seen, the distal opening 106 has reduced in size and the folded edge 124 is rolled inward.

In FIG. 3D, the proximal end 120 of the inner layer 108 has been moved further proximally and the distal end of the containing element 104 has been everted or inverted. A distal restriction 130 is shown where the folded edge 124 has rolled into the containing element 104. Through the steps shown herein, the outer layer 110 has remained relatively stationary relative to the vessel 128 and has remained opposed to the vessel 128. However, the folded edge 124 has rolled inward such that part of the outer layer 110 is now within itself. The folded edge 124 as defined by the meeting of the inner layer 108 and outer layer 110, forms the distal restriction 130 since the natural unbiased shape of the containing element 104 has about a 180-degree bend at the folded edge 124. Thus, when it is pulled inward, the folded edge 124 forms a shape shown in FIG. 3D which assists in closing off the clot within the interior chamber 114. In some implementations, the distal restriction 130 represents a complete closure where the interior chamber 114 is fully sealed, while in other implementations it represents merely a reduction in diameter. For example, the distal restriction can be a reduction from the nominal distal opening 106 size from and including about 5% to and including about 100%, from and including about 20% to and including about 90%, or about 80%. The distal restriction 130 does not necessarily need to be formed by the folded edge 124 and can be instead formed by other features, some of which will be shown in greater detail herein. Once the clot is contained within the interior chamber 114, the device 102 can be removed from the patient. In some implementations, the device can be removed through the lumen of the delivery catheter while keeping the delivery catheter in place. In other implementations, the entire system (e.g., the device 102, the catheter 126, and the delivery catheter) can be removed.

FIGS. 4A and 4B illustrate construction of an implementation of the device 102 in greater detail. Referring first to FIG. 4A, the outer layer 110 of the braid is shown as being unrolled or inverted about the folded edge 124. In this implementation, either the outer layer 110 or inner layer 108 or both may be constructed of a nitinol braid and have been shape set to a predetermined shape as shown in FIG. 1A. In the unrolled state, a membrane 112 may be applied to either or both of the outer layer 110 or the inner layer 108. For example, a dip or spray coating process may be used to apply a membrane 112 to the outer surface of the inner layer 108 as shown in FIG. 4A. The membrane 112 can extend anywhere between the folded edge 124 to the proximal end 120. In some implementations, the membrane 112 can be applied to the outer layer 110 only or in addition to the inner layer 108. By applying the membrane 112 to the outer layer 110 while it is unrolled, the membrane 112 would become adhered to the inner surface of the outer layer 110 when it is rolled back to its predetermined shape.

In some implementations, the membrane 112 can be laminated to the containing element 104 rather than dip or spray coated. In these implementations, the membrane 112 can be preformed out of an elastic material and then attached to either the inner layer 108 or outer layer 110 or both using any number of methods. For example, the membrane 112 can be formed by blow molding an extrusion of plastic such as a thermoplastic elastomer (TPE) or thermoplastic polyurethane (TPU) or a silicone or any other suitable polymeric material into the desired shape that generally can match a portion of the containing element 104 predetermined geometry. This can be done using medical balloon manufacturing techniques and materials that produce single wall thicknesses in the expanded region from and including about 0.0001″ to and including about 0.0020″, or about 0.0003″. Using this method, the distal end of the balloon may be cut off such that the membrane 112 has a distal opening that matches the profile of the containing element 104. In some of these implementations, the membrane 112 may be cut so that only the expanded portion remains and attaches to the expanded interior chamber 114 portion. The membrane 112 may additionally include a proximal end 120 that includes a tapered region 116 that matches the profile of the inner layer 108 or the outer layer 110. In certain implementations, the proximal end 120 of the membrane 112 may have a single wall thickness that is from and including about 0.0005″ to and including about 0.010″ or about 0.003″; however, this disclosure contemplates that any suitable membrane thickness may be used based on the corresponding application for the device 102. In some implementations, the membrane 112 can be formed using a blow molding process within the interior chamber 114 (i.e., the chamber defined between the inner layer 108 and the outer layer 110) such that the membrane 112 is adhered to the inner layer 108, outer layer 110, or permitted to “float” between the two layers during the manufacturing process. In some implementations, the membrane 112 can simply be an extruded tube or sheet that is manufactured to roughly the dimensions disclosed herein.

The membrane 112 can be attached to either the inner layer 108 or outer layer 110 or both in any number of ways. In some implementations, the membrane 112 is preformed and placed over the inner layer 108 when the containing element 104 is held in a configuration as shown in FIG. 4A. The membrane 112 can then be attached to the inner layer 108 by reflowing portions or all of it against the inner layer 108. For example, heat shrink, such as fluorinated ethylene propylene (FEP), can be used to compress the membrane 112 onto the inner layer 108 with heat and therefore cause the membrane 112 to melt into the inner layer 108 and become attached. In other implementations, a metal or plastic mold can be used to squeeze the membrane 112 against the inner layer 108. In some implementations, a heated element such as a mandrel can be placed within the interior chamber 114 and apply heat to the braid while the membrane 112 is held against the outer surface of the inner layer 108. In other implementations, the membrane 112 can be sandwiched between the inner layer 108 and outer layer 110 and heat can be applied to melt the membrane 112 into either or both of the layers. In still other implementations, the entire length of the membrane 112 does not necessarily need to be attached to the containing element 104. For example, only the distal end of the membrane 112 may be attached to the containing element 104. This may beneficially allow the containing element 104 to increase in length as it constricts within a delivery catheter without necessarily lengthening the entire membrane 112. This may reduce the radial opening force of the containing element 104 and thereby reduce friction within the delivery catheter. Alternatively, the membrane 112 may be attached at the distal end and proximal end but not along its center portion.

In some implementations, the membrane 112 can be made of multiple pieces of material and attached at various locations. For example, a cylindrical membrane 112 can be attached to the outer surface of the inner layer 108 while a funneled piece could be attached to the tapered region 116 of the outer layer 110. Any number of other connection locations, methods, and combinations of the membrane 112 are contemplated herein.

In FIG. 4B, the containing element 104 is shown with the outer layer 110 rolled back over the inner layer 108 after the membrane has been applied. As discussed herein, the membrane 112 can be attached to the inner layer 108 before this step or can be attached after this step, possibly with the addition of heat to reflow the membrane 112 into either the inner layer 108 or outer layer 110 or both. Once formed as shown in FIG. 4B, device 102 may be coupled to a catheter or similar device, such as the catheter 126 of FIG. 2, and control body. For example, a proximal portion of the outer layer 110 may be attached to an inner or outer surface of the catheter 126 and a proximal portion of the inner layer 108 may be attached to a longitudinally movable control body (e.g., a wire, tube, etc.) extending through and movable within the catheter 126.

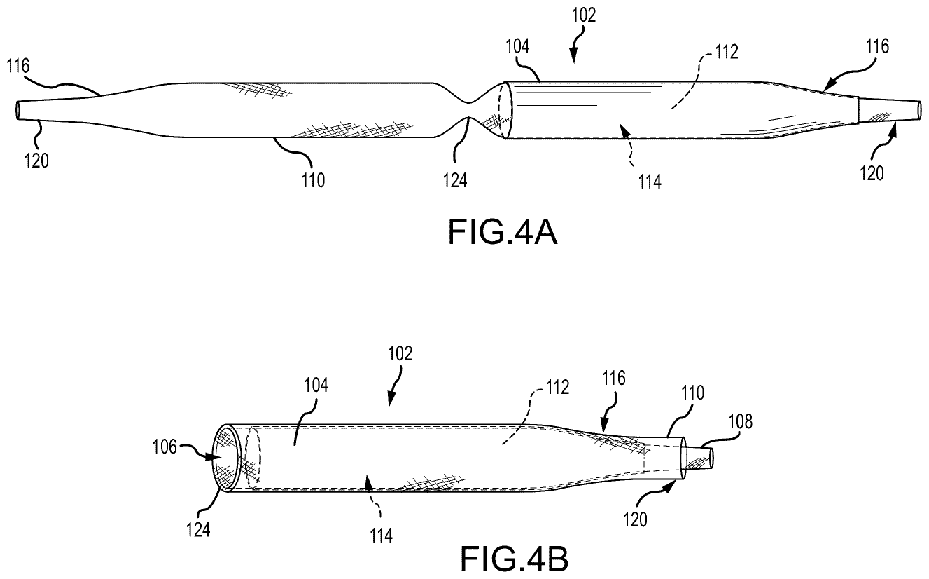

In FIG. 5, an alternate implementation of the distal restriction 130 is shown. The inner layer 108 is arranged such that it does not have the same profile as the outer layer 110 for at least a portion of the containing element 104. At the distal opening 106, the inner layer 108 forms a funnel that tapers back to a distal restriction 130 and then opens to the interior chamber 114. Such a shape may be achieved, e.g., by disposing containing element 104 about one or more correspondingly shaped mandrels or within a corresponding mold and setting (e.g., heat setting) the containing element 104 based on the material of containing element 104. In the implementation shown in FIG. 5, the distal restriction 130 is part of the profile of the containing element 104 in the expanded configuration and can be used as a passive valve that allows the entrance of clot 132 through it but prevents the escape of clot 132 from the interior chamber 114. For example, aspiration that is applied to the catheter 126 can urge the clot 132 material into distal opening 106. The proximally directed force on the clot 132 from the aspiration may allow the distal restriction 130 to open partly or entirely such that the clot 132 enters the interior chamber 114. Once the clot 132 is within the interior chamber 114 the distal restriction 130 can automatically return to its predetermined shape and close such that the clot 132 does not come out. The distal restriction 130 therefore acts like a one-way valve allowing clot 132 to come in but not leave. Any number of profiles are contemplated for this implementation of the distal restriction 130 including multiple distal restrictions and various tapered angles. In some implementations, the proximal end 120 of the inner layer 108 can be manipulated as described herein to change the shape of the distal restriction 130 further such as actively opening the containing element 104 and then letting it automatically close.

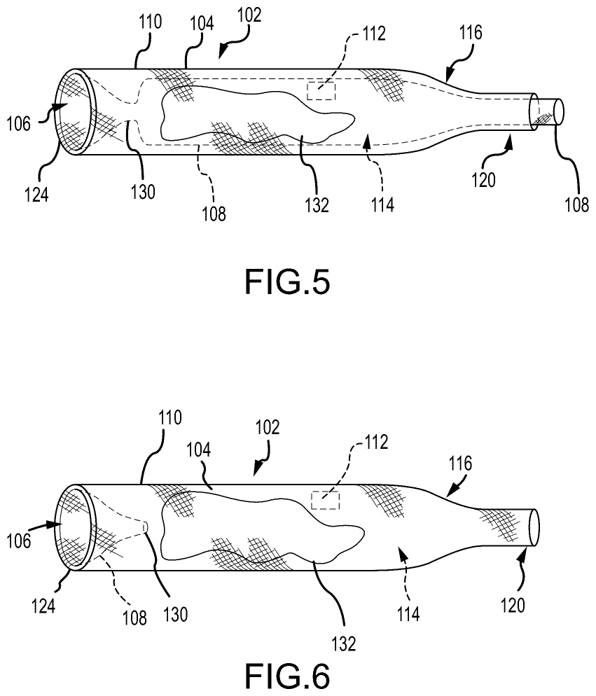

In FIG. 6, another implementation of the distal restriction 130 is shown where the inner layer 108 does not continue for the entire length of the containing element 104. In the implementation shown, the inner layer 108 forms a similar tapered entrance with a distal restriction 130 as discussed above but does not continue proximally beyond that. This may make the distal restriction 130 open easier to allow clot 132 to enter. In other implementations, the inner layer 108 can have any number of other profiles while not continuing through the entire length of the containing element. For example, the inner layer 108 can have a more or less 180-degree folded edge as shown in FIG. 1A but only extends into the interior chamber 114 about halfway. Any number of other implementations with shorter inner layers 8 are contemplated.

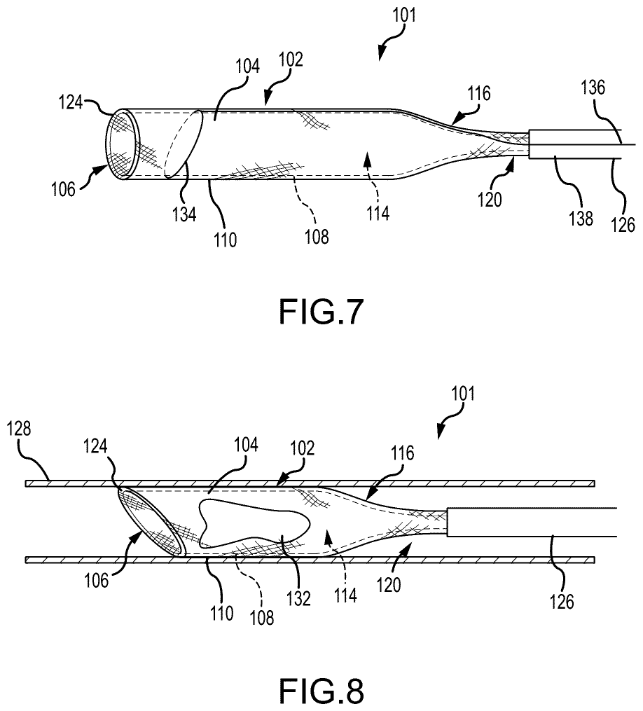

In FIG. 7, an implementation of the device 102 is shown with a filament 134 in the distal portion of the containing element 104. The filament 134 wraps around the distal end of the inner layer 108 and can be used to actively create a distal restriction 130 by cinching the inner layer 108 like a purse string or otherwise closing it by applying a proximally directed force. In certain implementations, the filament 134 may be positioned between the inner layer 108 and outer layer 110 such that it is not exposed within the interior chamber 114. Among other advantages, this may keep the interior chamber 114 and the distal opening 106 free of components which may block the entrance of the clot 132 into the interior chamber 114. The filament 134 may be connected to a distal control body 136 that runs along the length of the containing element 104 between the inner layer 108 and outer layer 110. The distal control body 136 can enter the interior chamber 114 or lumen of the catheter 126. The distal control body 136 can be used to actively create the distal restriction 130. In other implementations, the distal control body 136 can be used to apply a distally directed force on the containing element 104 during delivery that elongates the containing element 104 or reduces its diameter. The filament 134 shown in FIG. 7 sits at an angle within the inner layer 108 and outer layer 110. This may allow the filament 134 to accommodate a wider range of vessel diameters such that its angle increase or decreases depending on the vessel diameter. Nevertheless, this disclosure contemplates that the filament 134 can instead be routed about a longitudinal axis of the containing element 104. The filament 134 can be attached to the containing element 104 in any number of ways. It can be woven through the inner layer 108 or outer layer 110 or both. Alternatively, the inner layer 108 can have features such as bumps or restrictions that the filament can be positioned against. In other implementations, the filament 134 or distal control body 136 can be attached to the inner layer 108 using the membrane 112. For example, the membrane 112 can be reflowed onto the inner layer 108 and at the same time the filament 134 can be within the membrane 112 such that it is secured against the inner layer 108 at one or more areas. The device 102 includes a delivery catheter 138 shown that the containing element 104 extends out of into a vessel 128.

While FIG. 7 illustrates a device including a single filament that extends about the entirety of the containing element 104 and forms loop that is biased relative to a longitudinal axis of the containing element 104. This disclosure contemplates other arrangements and configurations related to the filament 134. For example, this disclosure contemplates that the filament 134 may form a loop that is substantially perpendicular to the longitudinal axis of containing element 104. This disclosure also contemplates that the filament 134 may not form a complete loop but extend about only a portion of the containing element 104. Finally, this disclosure also contemplates that the device 102 may include multiple filaments, with the filaments being independently controllable (e.g., coupled to respective control bodies) or simultaneously controllable (e.g., coupled to a common control body).

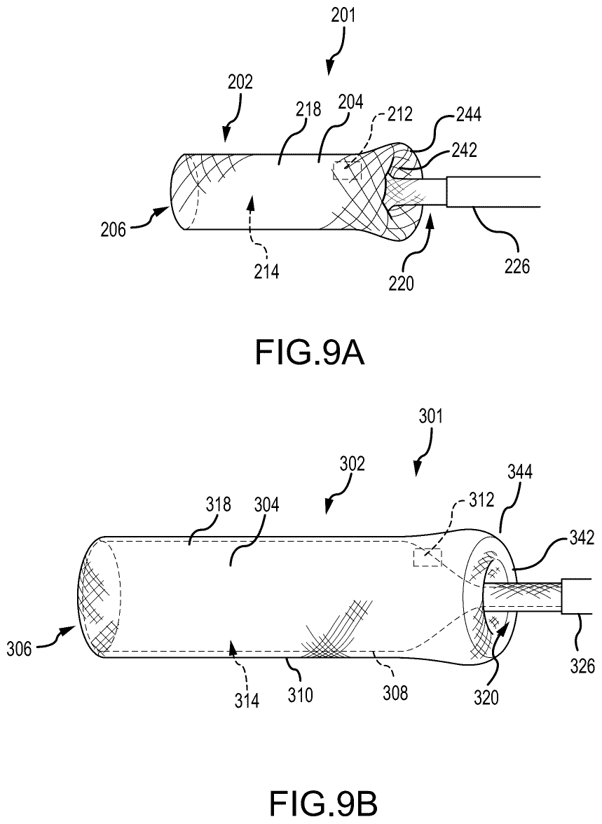

In FIG. 8, an alternate implementation of the device 102 is shown with an angled distal opening 106. Among other advantages, the angled distal opening 106 may allow the containing element 104 to accommodate a wider range of vessel diameters since the angle of the distal opening 106 can increase or decrease depending on the size of the vessel 128. This can allow the distal opening 106 to remain as patent as possible in its expanded configuration.

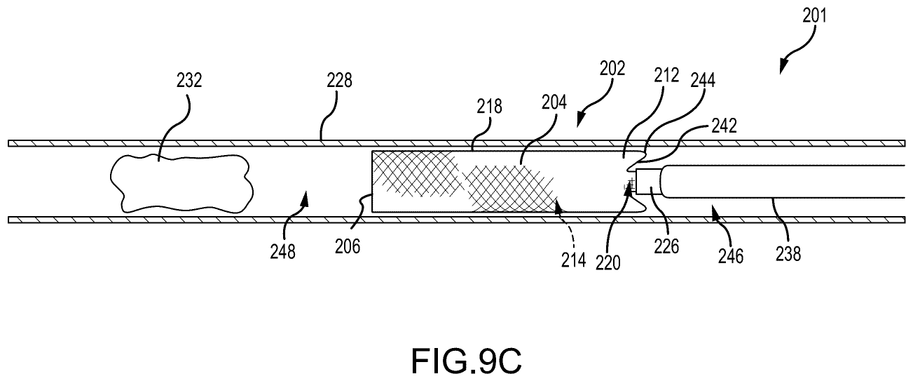

FIGS. 9A and 9B illustrate alternate devices according to this disclosure. Specifically, FIG. 9A illustrates a tool 201 including a device 202 coupled to a catheter 226. The device 202 includes a vessel sealing surface 244 and a flow restricting surfaces 242. The device 202 has similar elements as device 102, including a containing element 204, a distal opening 206, a membrane 212, an expandable interior chamber 214, an outer wall portion 218, a proximal end 220, and a catheter 226. Similarly, FIG. 9B illustrates a device 302 with a vessel sealing surface 344 and a flow restricting surfaces 342. The device 302 has similar elements as the device 102 and the device 202, including a containing element 304, a distal opening 306, a membrane 312, an expandable interior chamber 314, an outer wall portion 318, a proximal end 320, and a catheter 326.

As shown in FIG. 9A, the outer wall portion 218 of the device 202 and the flow restricting surfaces 242 are formed using a single braided layer coupled to a membrane 212. In contrast, FIG. 9B illustrates a tool 301 including a device 302 coupled to a catheter 326 in which the membrane 312 of the device 302 is disposed between the inner layer 308 and the outer layer 310. The device 202 of FIG.

In the case of the device 202 of FIG. 9A, the flow restricting surface 242 is formed by a layer of the containing element 204 that folds back as it expands radially and then forms the vessel sealing surface 244 at the proximal rim of the containing element 204. A membrane 212 on the flow restricting surface 242 limits or prevents altogether fluid from flowing through flow restricting surface 242. The membrane 212 can exist on the containing element 204 from the catheter 226 to the distal opening 206. Alternatively, the membrane 212 can exist for a subregion of the device 202 such as the flow restricting surface 242.

In the case of the device 302 of FIG. 9B, the device 302 includes an inner layer 308 and an outer layer 310 and the membrane 312 may disposed on one layer (e.g., the inner layer 308), as previously discussed in this disclosure. During use, the membrane 312 may be useful in forming a seal at the vessel sealing surface 344 between the fluidic path within the catheter 326 and the vessel 328 such that distally directed blood flow in the vessel cannot pass the flow restricting surface 342.

In each of the device 202 and the device 302, the shape of the flow restricting surface (the surface 242 for the device 202 and the surfaces 342 for the device 302) can be proximally concave as shown in FIGS. 9A and 9B, respectively. The concavity can be curved like a cup or can be a gradual taper or any other suitable geometries to restrict flow and seal along the lumen of the vessel as will be shown in more detail. This configuration is particularly useful for blocking proximal flow that is directed along the length of the catheter toward the containing element. A positive pressure gradient across the flow restricting surface causes the vessel sealing surface to seal against the vessel lumen. This may include expansion of the vessel sealing surface due to the pressure gradient.

In FIG. 9C, the device 202 of FIG. 9A is shown deployed in a vessel 228 proximal to a clot 232. As shown, the flow restricting surface 242 folds back on itself to create at least one proximally concave portion, which can create a higher resistance to distally directed blood flow within the vessel 228 as will be described in more detail below. The containing element 204 can be delivered through a delivery catheter 238. The vessel sealing surface 244 is shown pressing against the vessel 228 luminal walls and forms a seal that prevents blood flow. In this configuration, two distinct fluidic regions are created and separated at least partially by the flow restricting surface 242. With reference to the orientation shown in FIG. 9C, around the catheters and to the right of the flow restricting surface 242 is a first fluid region 246, and to the left of the flow restricting surface 242 is a second fluid region 248. The second fluid region 248 includes the interior chamber 214, the inner lumen of the catheter 226 and the lumen of the vessel 228 that is to the left of the containing element 204. If the pressure within the first fluid region 246 is equal to or higher than the pressure of the second fluid region, the vessel sealing surface 244 will seal against the vessel 228 and flow can be restricted by the flow restricting surface 242. In some implementations, the flow restricting surface 242 can be entirely impermeable so that there is no fluid communication between the two fluid regions. This can occur due to positive blood pressure or blood flow within the first fluid region 246 or a negative pressure within the second fluid region 248 due to vacuum applied to the catheter 226. In general, such functionality is like a one-way valve or leaflet valve where the inversion of the flow restricting surface 242 creates a region where vacuum pressure pushes the outer wall portion 218 radially outward.

Flow restricting surfaces as shown in FIGS. 9A and 9B, may be integrated into the device 102 shown in FIG. 1A in place of the tapered region 116. While the device 102 as shown in FIG. 1A similarly forms two fluid regions, the first fluid region (i.e., the region distal the catheter 126 and including the internal volume of the containing element 104) will have an equal or lower pressure than the second fluid region (i.e., the region proximal the containing element 104 and surrounding the tapered region 116), thereby forming a zero or negative pressure gradient across the wall of the containing element 104. As the pressure gradient increases and the forces applied to the tapered region 116 exceed the radial expansion force of the outer wall portion 118, the outer wall portion 118 may collapse and allow blood flow to go between the vessel 128 and the outer wall portion 118. Accordingly, when the device 102 includes the tapered region 116, the amount of negative pressure that the first fluid region can achieve and/or the pressure gradient across the containing element 104 may be limited and may prevent clot or other material from being withdrawn into the containing element 104 since the collapsing outer wall portion 118 reduces the diameter of the interior chamber 114. Specifically, when a clot 132 is at the distal opening 106 and negative pressure begins to build within the first fluid region, the interior chamber 114 may collapse, increasing the difficulty of drawing the clot into the interior chamber 114. As negative pressure continues to build within the interior chamber 114, the interior chamber 114 may further collapse such that the clot 132 cannot be completely drawn into the containing element 104. Accordingly, including a proximally concave flow restriction surface, such as those of device 202 and device 302, can increase the operating pressure gradient of the containing element 104 of the device 102 and improve clot ingestion.

Referring back to FIG. 9C, aspiration can be applied to the catheter 226 to ingest the clot 232 into the containing element 204. As negative pressure builds within the second fluid region 248, the vessel sealing surface 244 expands and seals against the vessel 228 and therefore blocks fluid flow coming from the first fluid region 246. This allows the negative pressure to continue building in the second fluid region 248 and it also prevents the outer wall portion 218 of containing element 204 from collapsing since there is no fluid flow going between the vessel 228 and the outer wall portion 218. The clot 232 can therefore be aspirated into the containing element 204 or pulled in with a stent retriever while blood flow is blocked by the device 202.

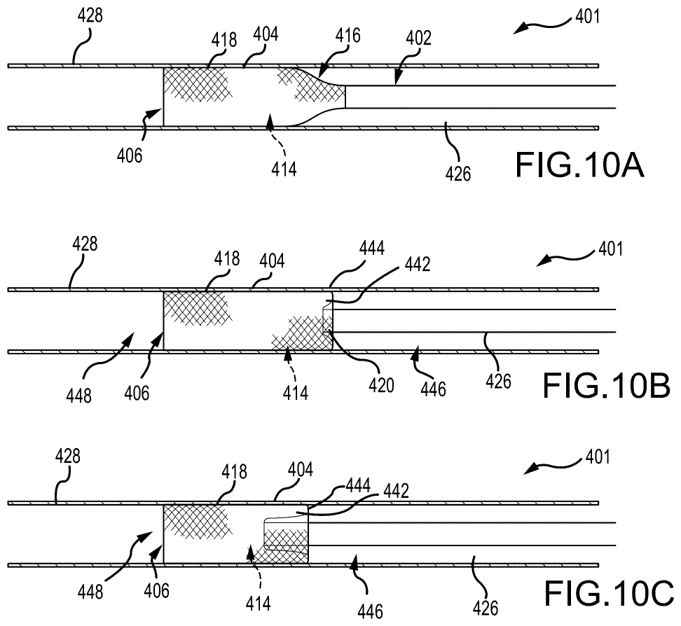

In FIGS. 10A-10C, an alternate implementation of a tool 401 including a device 402 according to this disclosure is shown, the device 402 including a flow restricting surface 442. In FIG. 10A, the device 402 is deployed within a vessel 428. As with the device 102 of FIG. 1A, the device 402 includes a tapered region 416 where an outer wall portion 418 gradually tapers from a proximal end 420 to an expanded portion that is against the wall of the vessel 428. The tapered region 416 forms an acute angle relative to the longitudinal axis of a containing element 404 of the device 402, meaning the angle formed is from about and including 0 degrees to about and including 90 degrees. As previously mentioned, when vacuum is applied via a catheter 426 in fluid communication with an interior chamber 414 of the containing element 404 (or a pressure gradient is otherwise formed across the wall of the containing element 404), the expanded outer wall portion 418 may collapse due to the negative pressure within the containing element 404 and the relative positive pressure in the vessel 428 proximal the containing element 404. This is further compounded when the proximal end of the vessel 428 includes normal blood pressure from about and including 50 mmHG to about and including 200 mmHg. However, even in instances where the blood pressure within the proximal end of the vessel 428 is zero, a negative pressure within the interior chamber 414 (e.g., due to aspiration) creates a pressure gradient across the outer wall portion 418 and the tapered region 416 that induces an inward radial pressure on the containing element 404 and that may cause the containing element 404 to collapse. As previously noted, this may prevent a clot or other target material from entering the interior chamber 414 due to the interior chamber 414 being collapsed and may also limit the amount of negative pressure that can be delivered to the vessel 428.

One solution to this potential problem is to increase the radial self-expanding force of the containing element 404 so that even under high vacuum pressures it does not collapse. However, too high of a self-expanding force may damage the vessel 428 or make delivery through the delivery catheter too difficult. Another solution is to use other mechanisms or devices (such as stent retrievers or other control elements) to keep the containing element 404 patent, but these may be complicated or difficult to adjust.

A third solution is illustrated in FIG. 10B and includes sealing of the flow restricting surface 442 with a vessel surface 444. More specifically, in FIG. 10B, the containing element 404 is configured to transition its tapered region 416 to a flow restricting surface 442. This can be accomplished by moving the delivery catheter forward while the containing element 404 is held in place at least partially by the wall of the vessel 428. Doing so results in formation of the flow restricting surface 442 (e.g., the proximally concave shape illustrated in FIGS. 9A and 9B) and a corresponding vessel sealing surface 444. In certain instances, the seal may have an appearance similar to an umbrella or diaphragm valve. The flow restricting surface 442 includes at least one section where the angle between it and the longitudinal axis of the containing element 104 is obtuse meaning between about and including 90 degrees to about and including 180 degrees. The flow restricting surface 442 may also not be a single angle but may be a curved surface such as a cup or any other shape.

As shown in FIG. 10B, the flow restricting surface 442 delineates a first fluid region 446 to the right and a second fluid region 448 to the left. When negative pressure is applied within the interior chamber 414, the pressure gradient between the first fluid region 446 and the second fluid region 448 now pushes the flow restricting surface 442 radially outward thereby preventing the containing element 404 from collapsing. By doing so, an improved seal is formed within the vessel 428 at the vessel sealing surface 444 that prevents blood from the proximal end of the vessel 428 from moving past the flow restricting surface 442.

In FIG. 10C, the flow restricting surface 442 is increased by moving the delivery catheter further distally. Both configurations of the device in FIG. 10B and FIG. 10C may be sufficient for preventing collapse of the containing element 404 during a positive pressure gradient between the first fluid region 446 and the second fluid region 448.

In some implementations, the flow restricting surface is formed by device manipulation. For example, as shown in FIGS. 10A-10C, the user or a mechanism can advance the delivery catheter forward which forms the flow restricting surface 442. Other portions of the device such as the catheter may be similarly used. Other components such as control wires or catheters may be similarly used to form the flow restricting surface by converting the tapered region into the flow restricting surface, e.g., by “popping” the tapered region inward. In still other implementations, the flow restricting surface can be at least partially predetermined within the shape of the containing element. For example, in FIG. 9A the flow restricting surface 242 is formed during manufacturing such that its unconstrained shape includes a folded back flow restricting surface 242. This may be accomplished by any number of manufacturing methods such as shape setting a nitinol braid into the desired shape and dip coating a membrane material onto the braid in that shape. In other implementations, the predetermined shape may be close but not quite a folded back flow restricting surface such as an angle that is about and including 25 to about and including 90 degrees, or from about and including 30 to about and including 60 degrees, or about 45 degrees. In such implementations, the predetermined shape by itself does not create a folded back flow restricting surface but does facilitate manipulation of the device to create the folded back portion more readily. In certain implementations, the flow restricting surface may include two bends with a straight portion between that forms an angle with the longitudinal axis. Alternatively, the flow restricting surface may include two larger bends that are connected. The flow restricting surface may include any number of bends including more than two bends. When the containing element moves through the delivery catheter the folded back portion may be folded back in a collapsed state or may be unfolded such that the flow restricting surface is formed when it is deployed from the delivery catheter.

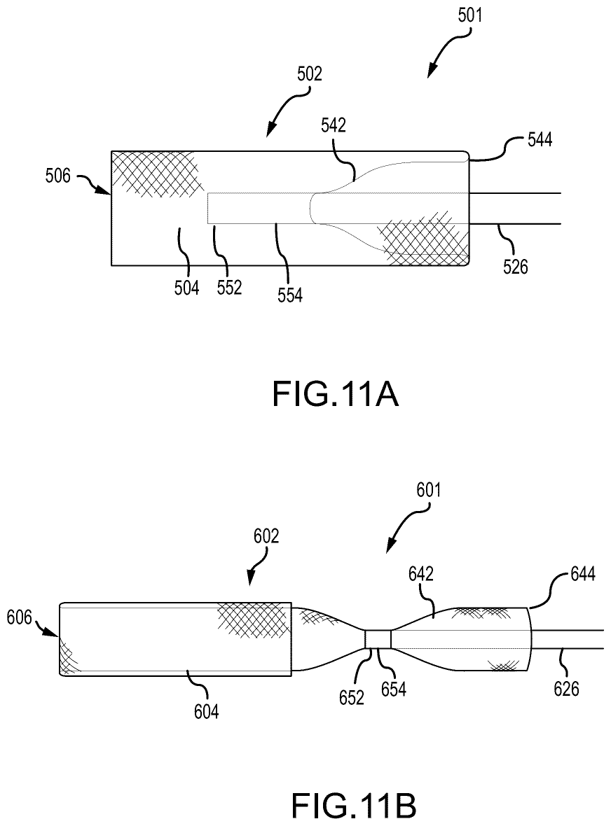

In FIGS. 11A and 11B, are alternate implementations of devices according to the present disclosure that include both flow restricting surfaces and containing elements. FIG. 11A illustrates a tool 501 including a device 502 having a containing element 504 with an expandable flow restricting surface 542 and a distal opening 506. The flow restricting surface 542 forms a flow restricting connection 554 to the outside of a catheter 526 of the device 502 and then expands backward along the length of the catheter 526. In contrast, in FIG. 9A, the flow restricting surface 242 first extends forward before expanding and folding backward. As shown in FIG. 11A, the catheter tip 552 of the device 502 therefore may extend into the containing element 504. The flow restricting surface 542 is also a more acute angle relative to the centerline of the catheter 526 than the embodiment illustrated in FIG. 9A. As shown, the device 502 forms a vessel sealing surface 544 by a very tight fold where it transitions from the flow restricting surface 542 to the outer wall portion 518.

FIG. 11B illustrates a tool 601 including a device 602 that forms an hourglass shape where a flow restricting surface 642 of the device 602 is separate from a containing element 604 (which is shown having a distal opening 606). The flow restricting surface 642 is an expandable surface that opens against the vessel and forms a vessel sealing surface 644 at its rim. The containing element 604 is then also connected to a catheter 626 and extends forward of the catheter 626 similar to other funnel shaped catheters described herein. In this embodiment, the flow restricting surface 642 acts to prevent fluid flow from going past it and therefore improves the function of the containing element 604 in removing tissue.

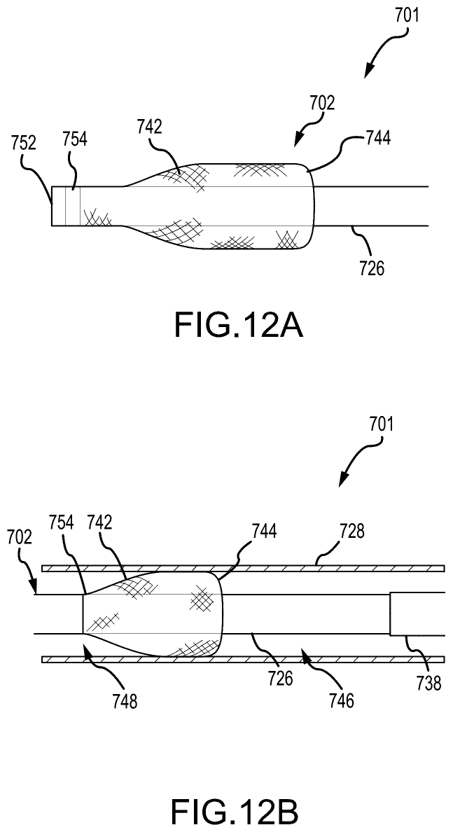

In FIG. 12A, a tool 701 including a device 702 is shown that includes a flow restricting surface 742 but not a container. In this embodiment, the flow restricting surface 742 can be used to block fluid flow past the flow restricting surface 742. This can be used to remove clot or other tissue from vessels as discussed by applying aspiration to the catheter 726.

In FIG. 12B, the device 702 is shown in a vessel 728 with the vessel sealing surface 744 against the vessel. A distal tip 752 of the catheter 726 extends through the flow restricting surface 742 which forms a connection 754. The catheter 726 is illustrated as extending from a delivery catheter 738. The flow restricting surface 742 separates a first fluid region 746 and a second fluid region 748 as shown in FIG. 12B. Aspiration can be applied to the catheter 726 and by blocking the blood flow, the flow restricting surface 742 enables higher negative pressure within the second fluid region 748 which therefore can impart higher mobilization forces on the clot. Alternatively, other techniques such as stent retrievers can be used to grab the clot and the lack of blood pressure and flow in the second fluid region may improve efficacy or prevent the clot from fragmenting. In some embodiments, the flow restricting surface 742 can be placed directly in the Middle Cerebral Artery (MCA) such that other flow occlusion devices, like balloon guide catheters, are not required in the carotid arteries. In other embodiments, the flow restricting surface 742 can be placed in the Internal Carotid Artery (ICA) while the distal tip 752 of the catheter is advanced to the MCA and the device 102 therefore performs the same function as a balloon guide catheter but in a smaller comparable size. In still other embodiments, the device 702 can be used for other tissue removal applications such as deep vein thrombosis, pulmonary embolism, arterial thrombosis, stone removal, and the like. In still other embodiments, the device 702 can be used in any application requiring flow restriction.

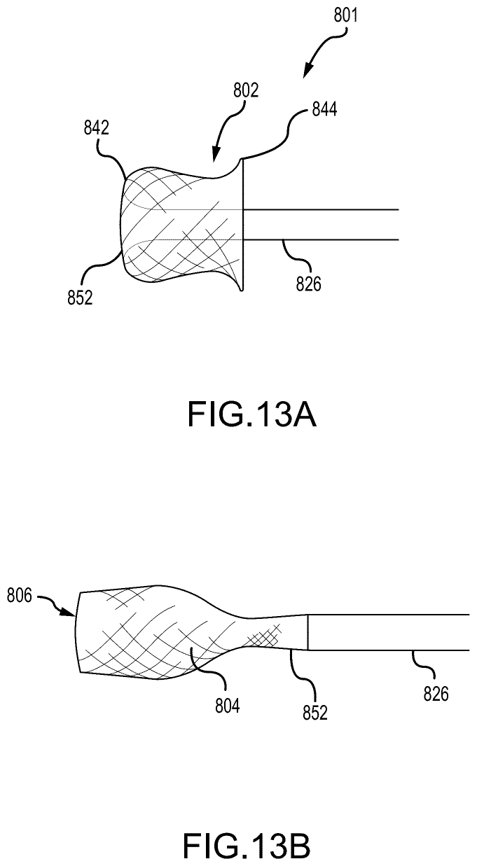

In FIGS. 13A and 13B, an alternate implementation of a tool 801 including a device 802 is shown. FIG. 13A illustrates the device 802 in a first state in which a flow restricting surface 842 forms a vessel sealing surface. FIG. 13B illustrates the device 802 in a second state in which the device 802 forms a containing element 804 having a distal opening 806. For example, the device 802 can be first used in the configuration shown in FIG. 13A to block flow in the vessel with the flow restricting surface 842. Once the clot is mobilized and at a distal tip 852 of a catheter 826 to which the device 802 is coupled, the device 802 can be withdrawn such that the flow restricting surface 842 unfolds and extends over/around the clot to form a containing element 804. The clot may then be partially or fully enclosed by the unfolding containing element 804 and can be withdrawn.

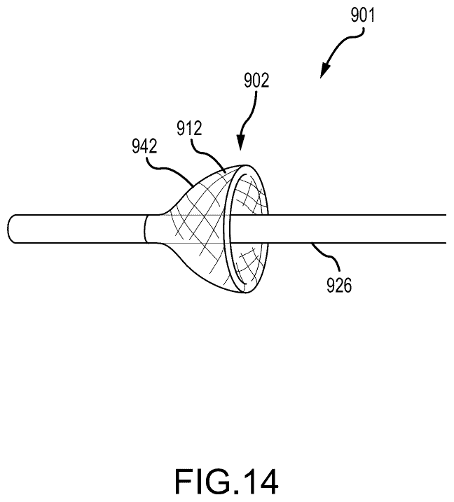

In FIG. 14, another alternate implementation of a device 902 is shown with a flow restricting surface 942 that can flip forward or backward. More specifically, the flow restricting surface 942 forms a bistable “cup” that can pop back and forth between a forward-facing cup and a backward-facing cup. The direction that the cup faces can be manipulated, e.g., by pulling a catheter 926 to which the device 902 is coupled forward or backward. When the cup faces backward, as shown in FIG. 14, the flow restricting surface 942 may block flow in the case of a positive pressure gradient where a first fluid region (e.g., on a proximal side of the flow restricting surface 942) has a higher pressure then a second fluid region (e.g., on a distal side of the flow restricting surface 942). When the cup faces forward, the flow restricting surface 942 may block flow in the case of a negative pressure gradient where the first fluid region has a lower pressure than the second fluid region. In this implementation, the user can adjust the device to achieve flow restriction in either direction. The flow restricting surface 942 may be formed by two layers of a braid which are coated with a membrane 912 and where each layer is connected to the catheter 926.

The following discussion highlights various design alternatives and concepts that may be incorporated into devices according to the present disclosure. Unless specifically noted, any of the following features may be integrated into embodiments of this disclosure or otherwise within the scope of the concepts described herein.

Returning to the details of the embodiments of devices according to this disclosure and described with referent to FIGS. 1-14, the containing element can have an overall length suited to its procedure. In embodiments where the containing element is used primarily to restrict blood flow, the containing element can be shorter and may only need to be long enough to ensure that it adequately opposes the vessel walls or provides a funnel for clot removal. In embodiments such as any thrombectomy where a clot is being extracted, the containing element may be sized to receive the clot and any additional elements such as graspers or stent retrievers. For example, the containing element may have a length from and including about 5 mm to and including about 100 mm or from and including about 20 mm to and including about 60 mm or about 35 mm. In some embodiments, the containing element can be relatively short and only provide flow arrest for an aspiration procedure with minimal clot containment. In other embodiments, the containing element can be longer to contain a clot with or without a stent retriever.

The base membrane can include any number of additional coating layers such as lubricious coatings to reduce friction such as hydrophobic coatings, hydrophilic coatings, silane, surface treatments, plasma vapor depositions, or any other suitable outer layer for friction reduction. The coating may further include drug eluting coatings to deliver therapeutic agents or radiopaque elements such as barium sulfate within its material composition.

In some embodiments, the inner layer and the outer layer do not necessarily need to be contiguous, meaning they do not have to be a single material that wraps around at a folded edge. For example, the inner layer and the outer layer could be separate components that are connected at some point in the device. The membrane may serve as a method of connecting the two layers. The distal ends of the layers may be cut end braids or may be atraumatic looped ends of braids or the membrane itself may form the distal opening such that the layers terminate before the distal opening but the membrane extends to the distal opening. In such an embodiment, the membrane may therefore be an atraumatic feature either by its geometry or its material properties that make it atraumatic to a vessel. In still other embodiments, the inner layer and the outer layer can be connected via a folded edge at the proximal end rather than at the distal opening.

In some embodiments, the outer wall portion may have more than two layers. For example, there may be an outer layer, an inner layer, and any number of middle layers from and including about 1 to and including about 10 or about 2. The middle layers can be of a similar construction to either the inner layer or the outer layer such as a braided nitinol mesh. In other embodiments, the middle layer can be made from a different construction such as a laser cut nitinol tube or any suitable other material.

In some embodiments, the outer wall portion and the flow restricting surface may be formed of by a laminated surface such as a laser cut nitinol tube laminated with a polymeric material. In this embodiment, the metal tube forms a scaffold that the material such as PTFE can be adhered to through a lamination process.

In some embodiments, the flow restriction achieved by the containing element does not need to be binary, meaning on or off. The flow restriction can be attenuated and controlled to achieve a desired flow rate, pressure, or blood supply. In such embodiments, the device can include a set of mechanisms that control the opening of the containing element using the filament. Electronic and control algorithms can be applied to achieve a user target for blood flow. This can be controlled manually by the user or can be done automatically such that a processer determines what the desired target is and then determines the appropriate amount of opening of the containing element and movement of the distal control body. In still other embodiments, the containing element can be opened automatically by a processer depending on the step of the procedure. For example, in certain steps of the procedure it may be desirable to have more or less flow than other steps. Blood flow is important for brain health and although an ischemic stroke generally decreases blood flow, restoring blood flow quickly is imperative. The device may automatically restore blood flow at given intervals rather than only restoring blood flow after the clot is fully contained. Variable flow arrest may additionally be advantageous during controlled endovascular embolization or sclerosant treatment of high flow arterio-venous malformations or fistulas. Additionally, the user-controlled re-establishment of flow may be used to gradually restore flow and therefore prevent reperfusion injury. For example, after a clot has been removed the containing element can be collapsed over a duration of time that allows gradual reperfusion of the ischemic tissue.

Taken together, the structural elements of the containing element, in some embodiments a braid, and the membrane create an expandable interior chamber formed by the outer wall portion. The containing element has an unbiased diameter that can be collapsed for delivery through the delivery catheter or through the patient's vasculature. When the containing element is within a vessel that is at least partially smaller than the outer diameter of the containing element, it can apply an outward expansion pressure on the vessel. The outward expansion pressure can be considered the radial force or pressure that maintains the patency of the containing element. The outward expansion pressure can be designed and predetermined such that a vacuum pressure within the interior chamber that is used to aspirate the clot does not significantly collapse the interior chamber. In physics, a perfect vacuum has a pressure of about 760 mmHg, so an outward expansion pressure above this number plus any blood pressure on the outer surface would ensure that the interior chamber would not collapse under the pressures of aspiration on the inner surface and blood pressure on the outer surface. During delivery through a delivery catheter, the outward expansion pressure creates frictional drag between the containing element and the catheter lumen, especially through tortuous curvature. Therefore, a lower outward expansion pressure of the containing element would reduce the force required to translate the containing element through the delivery catheter. Additionally, by applying a distally directed force at the distal portion of the containing element can reduce the outward expansion pressure during delivery. In some embodiments, the outward expansion pressure is at least about 300 mmHg to about and including 2000 mmHg, from about 600 mmHg to and including about 1200 mmHg, or about 900 mmHg. The outward expansion pressure can be adjusted by the construction of the braid including wire diameters, wire count, material type, etc., and by the membrane material and thickness.

The device may additionally have radiopaque characteristics such that it is visible using fluoroscopy. This may include the use of radiopaque material such as platinum, tungsten, gold, or any other suitable material. For example, the filament, control body, or the wire used in the braid of the containing element may made partly of Nitinol with a platinum core. Marker bands or other radiopaque components may also be secured to the device so that the user can ascertain the location of the device.

The filament can be a round wire of a constant diameter from and including about 5 μm to and including about 300 μm or from and including about 50 μm to and including about 150 μm or about 100 μm. Alternatively, the diameter of the wire can vary along the length of the filament and can be larger in some areas and smaller in others which may allow variable stiffness and flexibility of the filament at various points which may beneficially allow the wire to bend preferentially in certain areas. In other embodiments, the filament can be of other shapes and constructions than a round wire. The filament shown in FIG. 7 is a generally circular shape. The circular diameter of the filament may be any given size that is suited for its application. For example, for use within blood vessels the diameter may be from and including about 1 mm to and including about 50 mm depending on the vessel. For cerebral arteries, the diameter may be from and including about 2 mm to and including about 6 mm or from and including about 3 mm to and including about 5 mm. The shape of the filament 134 may be determined by the natural curvature of the filament. Alternatively, the shape of the filament may be at least partially predetermined by other means such as plastic deformation, shape setting of Nitinol, mechanical properties and relative stiffnesses of a composite of materials, or any other means. The profile of the filament can be generally circular with a diameter that generally matches the intended vessel diameter or slightly larger or slightly smaller than the vessel diameter. The profile can alternatively be non-circular and include any number of other shapes or features. The filament can be made partly of metals such as Nitinol, stainless steel, or the like. Alternatively, filament can be made partly of plastics such as suture, polyester, or the like.

In some embodiments, the filament can be larger than the distal opening of the containing element. In other embodiments, the user can adjust the size of the distal opening before or during the procedure. For example, the size and shape of the filament perimeter can be adjusted by pulling the filament in or out of the control body. By adjusting the filament shape, the device can accommodate different vessel sizes and ensure that the distal opening is fully opposed to the vessel wall while the clot is withdrawn into the interior chamber. Alternatively, the distal opening can be actively transitioned to a collapsed or expanded configuration by the user during navigation, delivery, and removal.

In some embodiments, the filament can be fixedly connected to the control body such that movement of the control body moves the filament connection and thereby pulls the filament. In other embodiments, the filament can move relative to the control body. When the clot is in the containing element and the user wishes to transition to a closed configuration, they can pull the filament relative to the control body such that the profile of the filament which was previously an open loop cinches like a purse string and closes. This can be a method of closing the distal opening of the containing element.

The filament may weave through all of the loops or only a portion of the loops. In some embodiments, the filament only weaves through from and including about 1 loop to and including about 128 loops or from and including about 2 loops to and including about 24 loops or about 4 loops.

This disclosure provides substantial description to thrombectomy procedures where clot is removed; however, devices and methods of this disclosure are not but that is not intended to be limited in scope to such procedures. In some embodiments, a device according to this disclosure may be used primarily for restricting blood flow in vessels. Such an embodiment may be used as a substitute for a balloon guide catheter during thrombectomy procedure. Alternatively, it may be used to limit blood flow when gluing or embolizing arteriovenous malformations (AVMs), high flow fistulas, or aneurysms. In other embodiments, the device can be deployed in the internal carotid artery (ICA) to restrict blood flow during any number of neurovascular procedures including thrombectomy or stenting procedures. In other embodiments, the device may be used to temporarily restrict blood flow during treatment of vascular perforation or vessel injury to stop life threatening hemorrhage. In this embodiment, the device may be used to restrict blood flow but not entirely arrest it such that extremities still receive some blood flow but the patient's blood loss is controlled within a safe limit.

Devices according to this disclosure can have a variety of shapes and sizes serving as a platform for any type of thrombectomy, embolectomy, or foreign body, calculi, or tissue removal in any part of the body or vessel. The devices may provide proximal support and purchase for placement of distal devices such as rheolytic catheters, suction devices, graspers, balloons such as a Fogarty balloon, wire snares, stent retrievers, etc. for any size tube or vessel including arteries, veins, ureters, airways, bile ducts, and hollow viscous for retrieval of material. The devices and methods described within may be used in any number of other surgical procedure. For example, peripheral blood clots may be likewise removed with such a system. This could include but not limited to deep venous thrombosis, pulmonary emboli, clotted hemodialysis grafts or peripheral arterial thromboemboli, including the mesenteric and peripheral vascular tree. The expanded device provides the operator an anchor and purchase to the desired vascular tree for further intervention as needed.

Any number of other suitable applications may use such a device for contained removal of a tissue, foreign body, calculi, or other objects within a tubular contained space or even within non-tubular or non-contained spaces.

In some embodiments, devices according to this disclosure may contain all or only a portion of the various embodiments described herein. For example, the device may include a clot engagement element such as a stent retriever or aspiration catheter. Alternatively, the device may only include a containing element and a filament and the device may be used with an existing off-the-shelf available stent retriever. In such an embodiment, the containing element and filament may be sized to accept such a retriever. The device may be inserted into the body after the stent retriever has been deployed and captured the clot, potentially utilizing a Rapid Exchange technique. In this way it is a stand-alone system for capturing the clot that includes using other clot engagement elements. Any number of other configurations of the devices described herein are contemplated.

The devices and methods described herein can be used for any number of clinical applications where local flow arrest or clot removal or clot containment are desired. For example, the invention may be used for removing clot from a cerebral or carotid artery to treat an acute ischemic stroke. It may also be used for treatment of pulmonary embolisms, deep vein thrombosis (DVT) both chronic and acute, arterial thrombectomy, stone removal, blocking flow during radioactive fluid injections or selective embolization maneuvers, and cerebral venous sinus thrombosis. The device could be deployed in any number of vascular targets such as veins and arteries. A particular advantage of the invented device and method is that aspiration can be performed using a relatively small, catheter 126 or delivery catheter 138 where the containing element 104 creates a large lumen at the front of the catheter and fully contains the clot during removal from the patient.

The names and labels applied to the various components and parts should not be considered limiting to the scope of the invented device and method. For example, the term filament used herein may be interchangeably used with snare, wire, ribbon, coil, elongate member, or any other suitable term. The term catheter is used to describe an elongate member with a distal and proximal end with a lumen extending there through. The terms intermediate catheter, delivery catheter, filament catheter, guide catheter, and micro catheter may often be used interchangeably. The term container may often be interchangeably used with bag, containing element, container element, pouch, or any other suitable term. When referring to the opening of the distal opening, the terms releasing, deploying, opening, and expanding may be used interchangeably. When referring to the closure of the distal opening the terms cinching, closing, constraining, collapsing, constricting, snaring, or any other suitable term may often be used interchangeably. When referring to the radial constraining of the containing element by catheters, vessels, or filaments, the terms constraining, restricting, containing, collapsing, or constricting may also often be used interchangeably. The term distal or distal portion generally refers to areas of the device situated away from the center of the device in the direction of blood flow while the term proximal generally refers to areas of the device situated away from the center of the device in the opposite direction of blood flow. The term distal opening can refer to the distal portion of the containing element within about 1-10 mm of the distal most aspect.

Although embodiments of various methods and devices are described herein in detail with reference to certain versions, it should be appreciated that other versions, embodiments, methods of use, and combinations thereof are also possible. Therefore, the spirit and scope of the appended claims should not be limited to the description of the embodiments contained herein.

What is claimed is:

1. A device comprising:
a catheter having a distal catheter end and defining a catheter lumen;
a containing element coupled to the catheter, wherein the containing element is configured to be deployed from the distal catheter end of the catheter and includes an outer wall portion including:
an inner layer defining an inner chamber in communication with the catheter lumen; and
an outer layer coupled to the inner layer such that, when the containing element is deployed from the catheter, the inner layer and the outer layer form a distal folded edge defining a distal opening of the containing element in communication with the inner chamber; and
a control element extending through the catheter lumen and coupled to the containing element, wherein the control element is longitudinally movable relative to the catheter to selectively collapse the inner chamber defined by the inner layer when the containing element is deployed.

2. The device of claim 1, wherein the containing element is formed from a braided shape settable material.

3. The device of claim 1, wherein the containing element is coupled to the catheter by the outer layer.

4. The device of claim 1, wherein the outer wall portion further includes a membrane configured to prevent fluid flow through at least a portion of the outer wall portion.

5. The device of claim 4, wherein the membrane is disposed between the inner layer and the outer layer.

6. The device of claim 4, wherein the membrane is coupled to one the outer layer and the inner layer.

7. The device of claim 1, wherein the distal folded edge is configured to retain a predefined shape when deployed from the distal catheter end.

8. The device of claim 7, wherein the distal folded edge is formed by heat setting the containing element in a deployed shape.

9. The device of claim 1, wherein the control element is a control catheter coupled to a proximal portion of the inner layer.

10. The device of claim 1, wherein the control element is a filament extending between the inner layer and the outer layer of the outer wall portion.

11. The device of claim 1, wherein, when the containing element is deployed, the outer layer includes a proximal flow restriction portion.

12. The device of claim 11, wherein the proximal flow restriction portion is a proximally concave surface of the outer layer.

13. The device of claim 1, wherein the outer layer proximally tapers.

14. The device of claim 1, further comprising a delivery catheter within which the catheter is disposed.

15. A device comprising:
a catheter having a distal catheter end and defining a catheter lumen; and
a containing element coupled to the catheter, wherein the containing element is configured to be deployed from the distal catheter end of the catheter and includes an outer wall portion including:
an inner layer defining an inner chamber in communication with the catheter lumen; and
an outer layer coupled to the inner layer such that, when the containing element is deployed from the catheter, the inner layer and the outer layer form a distal folded edge defining a distal opening of the containing element in communication with the inner chamber;
wherein the outer layer is configured to form a proximally concave surface when the containing element is deployed from the distal catheter end.

16. The device of claim 15, wherein the containing element is formed from a braided shape settable material.

17. The device of claim 15, wherein the proximally concave surface includes a proximal folded edge and each of the distal folded edge and the proximal folded edge are configured to retain a predefined shape when deployed from the distal catheter end.

18. The device of claim 17, wherein the distal folded edge and the proximal folded edge are formed by heat setting the containing element in a deployed shape.

19. A system comprising:
an aspiration source;
a delivery catheter;
a catheter disposed within the delivery catheter, wherein the catheter includes a distal catheter end and defines a catheter lumen in fluid communication with the aspiration source;
a containing element coupled to the catheter, wherein the containing element is configured to be deployed from the distal catheter end of the catheter and includes an outer wall portion including:
an inner layer defining an inner chamber of the containing element in communication with the catheter lumen;
an outer layer coupled to the inner layer such that, when the containing element is deployed from the catheter, the inner layer and the outer layer form a distal folded edge defining a distal opening of the containing element in communication with the inner chamber; and
a membrane configured to restrict flow through the outer wall portion; and
a control element extending through the catheter lumen and coupled to the containing element, wherein the control element is longitudinally movable relative to the catheter to selectively collapse the inner chamber defined by the inner layer when the containing element is deployed.

20. The system of claim 19, wherein the outer layer is configured to form a proximally concave surface when the containing element is deployed from the distal catheter end.
